(19) United States
(12) Patent Application Publication
(10) Pub. No.: US 2024/0278020 A1
(43) Pub. Date: Aug. 22, 2024

(54) ELECTROCARDIOGRAM MORPHOLOGY EVALUATION MANAGEMENT IN IMPLANTABLE DEVICE

(71) Applicant: Impulse Dynamics NV, Willemstad (CW)

(72) Inventors: David PRUTCHI, Voorhees, NJ (US); Tamir BEN DAVID, Tel-Aviv (IL)

(73) Assignee: Impulse Dynamics NV, Willemstad (CW)

(21) Appl. No.: 18/415,753

(22) Filed: Jan. 18, 2024

Related U.S. Application Data
(60) Provisional application No. 63/447,365, filed on Feb. 22, 2023.

(51) Int. Cl. A61N 1/362; A61B 5/00; A61B 5/363

(52) U.S. Cl. CPC A61N 1/3621; A61B 5/363; A61B 5/686

(57) ABSTRACT

The present invention discloses means and methods for performing evaluation of an electrocardiogram (ECG) morphology in an implantable device, optionally the implantable device has a single ECG sensing lead.

### RELATED APPLICATIONS

This application claims the benefit of priority under 35 USC § 119(e) of U.S. Provisional Patent Application No. 63/447,365 filed on Feb. 22, 2023, the contents of which are incorporated by reference as if fully set forth herein in their entirety.

### FIELD AND BACKGROUND OF THE INVENTION

The present invention, in some embodiments thereof, relates to means and methods for evaluating an electrocardiogram (ECG) morphology in an implantable device and, more particularly, but not exclusively, to a means and methods for evaluating an electrocardiogram morphology in an implantable device having single ECG sensing lead.

Implantable devices are typically characterized with limited energy and processing resources, as a result of the need to miniature the device as much as possible. In some cardiac implantable devices, two or more monitoring and treatment modalities are combined using an ECG signal as input for managing the device. Such device configurations may include the following modalities:
Implantable cardioverter-defibrillator (ICD) combined with Cardiac resynchronization therapy (CRT);
Implantable cardioverter-defibrillator (ICD) combined with Cardiac contractility modulation;
Implantable cardioverter-defibrillator (ICD) combined with Cardiac contractility modulation and Cardiac resynchronization therapy (CRT).

Typically, such configurations use two or more ECG sensing leads as input for device management.

In some configurations, a single ECG sensing lead is used for inputs for all modalities. Typically, each treatment modality is provided at a specific conditions. ICD therapy is typically provided when cardiac arrhythmia is detected, Cardiac contractility modulation therapy is typically provided on a daily basis, during set time period (Cardiac contractility modulation on time period) and CRT is typically provided pending on heart rate (HR) value and Atrial-Ventricle (A-V) and Right ventricle-Left ventricle (V-V) timing delay values.

### SUMMARY OF THE INVENTION

Following is a non-exclusive list including some examples of embodiments of the invention. The invention also includes embodiments which include fewer than all the features in an example and embodiments using features from multiple examples, also if not expressly listed below.
Example 1. A method of performing morphology evaluation processes in an implantable device, the method comprising:
   a. providing instructions to said implantable device for performing a morphology evaluation process at a determined timing according to predetermined parameters;
   b. activating said morphology evaluation process for a predetermined period of time following sensing said predetermined parameters.
Example 2. The method according to example 1, wherein said predetermined parameters are one or more of a scheduled stimulation event and a tachycardia event.
Example 3. The method according to example 1 or example 2, wherein said scheduled stimulation are one or more of Cardiac contractility modulation and CRT.
Example 4. The method according to any one of examples 1-3, wherein when said predetermined parameters is a scheduled stimulation event, then said predetermined period of time is at least one selected form the group consisting of:
   a. up to 500 mS; and
   b. up to 50 mS.
Example 5. The method according to any one of examples 1-4, wherein said tachycardia event is defined when a heart rate is above 100 beats per minute (BPM).
Example 6. The method according to any one of examples 1-5, wherein when said predetermined parameters is a tachycardia event, then said predetermined period of time is as long as said tachycardia event is sensed.
Example 7. The method according to any one of examples 1-6, wherein when said predetermined parameters is a tachycardia event, then said predetermined period of time is of equal or greater than 1000 mS.
Example 8. The method according to any one of examples 1-7, wherein said predetermined parameters are one or more of:
   a. a detected atrial-ventricle (A-V) timing delay above a predetermined threshold;
   b. a detected right ventricle-left ventricle (V-V) timing delay above a predetermined threshold; and
   c. a detected P-P delay that is different from an R-R delay.
Example 9. The method according to any one of examples 1-8, wherein when said predetermined parameters is one or more of:
   a. a detected atrial-ventricle (A-V) timing delay above a predetermined threshold;
   b. a detected right ventricle-left ventricle (V-V) timing delay above a predetermined threshold; and
   c. a detected P-P delay that is different from an R-R delay;
   then said predetermined period of time is of up to 500 mS or up to 100 mS.
Example 10. The method according to any one of examples 1-9, wherein:
   a. said predetermined threshold of said atrial-ventricle (A-V) timing delay is a sensed value above 180 mS;
   b. said predetermined threshold of said atrial-ventricle (A-V) timing delay is a change of more than 10 mS from baseline measured A-V delay;
   c. said predetermined threshold of said right ventricle-left ventricle (V-V) timing delay is a sensed value above 10 mS;
   d. said predetermined threshold of said right ventricle-left ventricle (V-V) timing delay is a change of more than 1 mS from baseline measured V-V delay.
Example 11. A system for performing morphology evaluation processes in an implantable device comprising an implantable controller, said controller configured to carry out the method of:
   a. performing a morphology evaluation process at a determined timing according to predetermined parameters;
   b. activating said morphology evaluation process for a predetermined period of time following sensing said predetermined parameters.
Example 12. The system according to example 11, wherein said predetermined parameters are one or more of a scheduled stimulation event and a tachycardia event.
Example 13. The system according to example 11 or example 12, wherein said scheduled stimulation are one or more of Cardiac contractility modulation and CRT.
Example 14. The system according to any one of examples 11-13, wherein when said predetermined parameters is a scheduled stimulation event, then said predetermined period of time is at least one selected form the group consisting of:
   a. up to 500 mS; and
   b. up to 50 mS.
Example 15. The system according to any one of examples 11-14, wherein said tachycardia event is defined when a heart rate is above 100 beats per minute (BPM).
Example 16. The system according to any one of examples 11-15, wherein when said predetermined parameters is a tachycardia event, then said predetermined period of time is as long as said tachycardia event is sensed.
Example 17. The system according to any one of examples 11-16, wherein when said predetermined parameters is a tachycardia event, then said predetermined period of time is of equal or greater than 1000 mS.
Example 18. The system according to any one of examples 11-17, wherein said predetermined parameters are one or more of:
   a. a detected atrial-ventricle (A-V) timing delay above a predetermined threshold;
   b. a detected right ventricle-left ventricle (V-V) timing delay above a predetermined threshold; and
   c. a detected P-P delay that is different from an R-R delay.
Example 19. The system according to any one of examples 11-18, wherein when said predetermined parameters is one or more of:
   a. a detected atrial-ventricle (A-V) timing delay above a predetermined threshold;
   b. a detected right ventricle-left ventricle (V-V) timing delay above a predetermined threshold; and
   c. a detected P-P delay that is different from an R-R delay;
   then said predetermined period of time is of up to 500 mS or up to 100 mS.
Example 20. The system according to any one of examples 11-19, wherein:
   a. said predetermined threshold of said atrial-ventricle (A-V) timing delay is a sensed value above 180 mS;
   b. said predetermined threshold of said atrial-ventricle (A-V) timing delay is a change of more than 10 mS from baseline measured A-V delay;
   c. said predetermined threshold of said right ventricle-left ventricle (V-V) timing delay is a sensed value above 10 mS;
   d. said predetermined threshold of said right ventricle-left ventricle (V-V) timing delay is a change of more than 1 mS from baseline measured V-V delay.
Example 21. The method according to any one of examples 1-20, further comprising sending result of said morphology evaluation process to an external device.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic representation of an exemplary implantable device, according to some embodiments of the invention; and

FIG. 2 is an exemplary method of performing morphology evaluation processes in an implantable device, according to some embodiments of the invention.

### DESCRIPTION OF SPECIFIC EMBODIMENTS OF THE INVENTION

The present invention, in some embodiments thereof, relates to means and methods for evaluating an electrocardiogram (ECG) morphology in an implantable device and, more particularly, but not exclusively, to a means and methods for evaluating an electrocardiogram morphology in an implantable device having a single ECG sensing lead.

### Overview

An aspect of some embodiments of the invention relates to monitoring a morphology of an ECG signal for managing an implantable device, for example an Implantable Cardioverter-Defibrillator (ICD). In some embodiments, the monitoring is performed by a control comprising instructions for monitoring one or more of the following parameters of the morphology monitoring process: the timing of morphology evaluation and the duration of the signal sampling window. In some embodiments, a potential advantage of utilizing these parameters during the monitoring process is that it potentially reduces the energy consumption of the device. In some embodiments, the morphology is assessed during one or more of the following events (or timings): during stimulation and when heart rate is within tachycardia periods (Tachycardia is typically set when HR is above 100 per minutes (BPM), for example from about 100 BPM to about 300 BPM. In some embodiments, the signal sampling window duration is done following one or more of the following rules: short sampling time window (up to 50 mS) during stimulation on predetermined time periods and larger time window of sampling (longer than 50 mS) during tachycardia periods (in some embodiments, sampling may be continuous during the whole period of tachycardia).

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

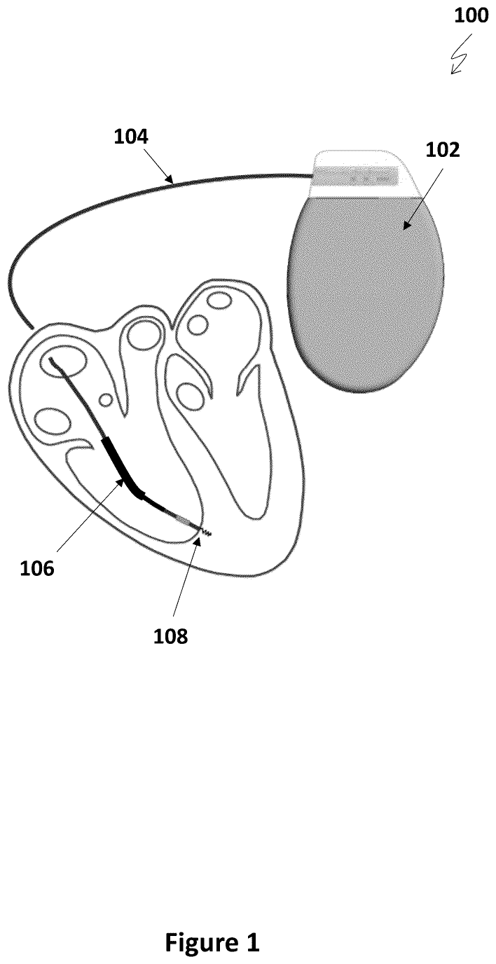

Referring now to FIG. 1, showing a schematic representation of an exemplary implantable device, according to some embodiments of the invention. In some embodiments, the implantable device 100 comprises a pulse generator comprising a housing 102 and one or more leads 104, in FIG. 1 only one lead 104 is shown, which are optionally couplable to the housing 102 of the device 100 with one or more connectors (not shown). In some embodiments, the housing 102 is implanted outside the heart, for example in the subclavian area. Optionally, implantation is via a minimally invasive procedure. In some embodiments, the housing 102 is implanted subcutaneously, in proximity of the left chest. In some embodiments, the housing 102 comprises within, all the necessary electronics and power source for the correct functioning of the implantable device 100, for example, a pulse generator is optionally used to generate the signal, for example, including a power circuitry, for example, including one or more storage capacitors. In some embodiments, the device 100 further comprises a ventricular detector used to detect atypical ventricular activation, which can be a contra-indication to signal application. In some embodiments, not shown in FIG. 1 since only one exemplary lead 104 is shown, the device 100 further comprises an atrial detector used to detect atypical atrial activation, which may be used as an input to decision making by device. In some embodiments, the device 100 further comprises one or more sensors configured to deliver one or more sensor input data, for example electrical sensors or other sensors, such as flow, pressure and/or acceleration sensors. In some embodiments, data from the sensors is optionally further processed (e.g., by a controller and/or detectors) and are optionally used as an input to decision making processes in device 100, which will be further explained below. In some embodiments, the device 100 further comprises a controller configured for executing one or more logics to decide, for example, a timing and/or other parameters of a signal and/or if a signal is to be applied, which will be further explained below. In some embodiments, the controller controls the applying of stimulation pulses according to the treatment plan. Optionally, the controller effects a change in the plan, for example so as to compensate for real time deviations from the treatment plan (e.g. a skipped stimulation). In some embodiments, the device 100 further comprises a memory configured for storing logic, past effects, therapeutic plans, adverse events and/or pulse parameters. In some embodiments, the controller and/or memory are programmed with one or more treatment plans (optionally set for the specific patient) and/or with one or more fallback treatment plans.

In some embodiments, the controller and/or memory are programmed with one or more instructions for monitoring a morphology of an ECG signal for managing the implantable device 100, for example an Implantable Cardioverter-Defibrillator (ICD).

In some embodiments, the device 100 further comprises a logger configured for storing activities of device 100 and/or of the patient. In some embodiments, such a log and/or programming may use a communication module to send data from device 100, for example, to a programmer (not shown) and/or to receive data, for example, programming, for example, pulse parameters.

Following the exemplary option that the implantable device 100 is for example an Implantable Cardioverter-Defibrillator (ICD), a same lead 104 is used for all the necessary activities, for example, monitoring the cardiac activity, providing Cardiac resynchronization therapy (CRT), providing Cardiac contractility modulation, and any combination thereof. In some embodiments, regarding the activation and functioning of the ICD, activation of the ICD is by an ICD module which includes or is connected to: ICD control, a defibrillation pulse generator (via one or more capacitors), a power source (e.g. a battery) and power source management circuitry, and ICD sensor which senses an applied pulse to verify the pulse is within a selected (e.g. programmed) amplitude and/or duration. In some embodiments, activation of cardiac contractility modulation is by a cardiac contractility modulation module which includes or is connected to: cardiac contractility modulation control, a cardiac contractility modulation generator. As mentioned before, in some embodiments, the ICD coil and one more electrodes for pacing and/or cardiac contractility modulation are configured on the same lead. In some embodiments, separate leads are used for the ICD coil and one more electrodes for pacing and/or cardiac contractility modulation.

In some embodiments, the lead 104 extends from the housing 102, and at least a distal segment of the lead is implanted within the heart, as schematically shown for example in FIG. 1. In some embodiments, the lead comprise an ICD defibrillation coil 106 located along the lead 104. In some embodiments, the lead 104 ends with a tip electrode 108. In some embodiments, the tip electrode may be configured as a contact electrode, a screw-in electrode, a sutured electrode, a free-floating electrode and/or other types. In some embodiments, a tip electrode is formed with a threading so as to be threaded into the tissue. Alternatively, a tip electrode is solely placed in contact with the tissue.

In some embodiments, the cardiac contractility modulation signal is applied to the heart during a relative and/or absolute refractory period of the heart. In some embodiments, the signal is selected to increase the contractility of a cardiac ventricle when the electric field of the signal stimulates such ventricular tissue, for example, the left ventricle, the right ventricle and/or a ventricular septum. In some embodiments of the invention, contractility modulation is provided by phosphorylation of phospholamban caused by the signal. In some embodiments of the invention, contractility modulation is caused by a change in protein transcription and/or mRNA creation caused by the signal, optionally in the form of reversal of a fetal gene program. It is noted that in some embodiments the cardiac contractility modulation signal may be excitatory to tissue other than that to which it is applied.

While not being limited to a single pulse sequence, the term cardiac contractility modulation is used to describe any of a family of signals which includes a significant component applied during an absolute refractory period and which has a clinically significant effect on cardiac contractility in an acute and/or chronic fashion and/or which causes a reversal of fetal gene programs and/or which increases phosphorylation of phospholamban. In some embodiments, the signal is potentially excitatory to one part of the heart but non-excitatory to other parts. For example, a signal can be excitatory in atria, but applied at a timing (relative to ventricular activation) when it is not excitatory in the ventricle.

In some embodiments of the invention, the signal while potentially stimulatory during the receptive period of the cardiac cycle, is non-excitatory due to its timing. In particular, the signal is applied during the refractory period of the tissue which is affected by it and, optionally, within the absolute refractory period.

In some embodiment, a device electrode, for example the cardiac contractility modulation applying electrode, is used for measuring the R wave amplitude and/or RR interval of the cardiac cycle.

As previously mentioned, in some embodiments, an exemplary implantable device 100 is characterized with limited energy and processing resources, as a result of the need to miniature the device 100 as much as possible. In some embodiments, additionally, the implantable device 100 comprises two or more monitoring and treatment modalities are combined for device management. In some embodiments, ECG signal is used as input for device management. In some embodiments, the device 100 comprise a configuration that includes one or more of the following modalities: Implantable cardioverter-defibrillator (ICD) combined with Cardiac resynchronization therapy (CRT); Implantable cardioverter-defibrillator (ICD) combined with Cardiac contractility modulation; and Implantable cardioverter-defibrillator (ICD) combined with Cardiac contractility modulation and Cardiac resynchronization therapy (CRT). In some embodiments, in view of these modalities, the device 100 comprises two or more ECG sensing leads as input for device management, or, as previously mentioned, the device 100 may use a single ECG sensing lead for inputs for all modalities.

In some embodiments, each treatment modality is provided at a specific conditions. For example, ICD therapy is provided when Ventricular arrhythmia is detected, Cardiac contractility modulation therapy is provided on a daily basis, during set time period (Cardiac contractility modulation on time period) and CRT is provided pending on heart rate (HR) value and Atrial-Ventricle (A-V) and Right ventricle-Left ventricle (V-V) timing delay values.

Therefore, in some embodiments, an exemplary device is characterized by being an implantable ICD device having at least one additional stimulation modality and having at least one lead that is used for one or more of stimulation, electrical shock and ECG measurement.

### Exemplary Monitoring of the ECG Morphology

In some embodiments, as explained above, the implantation device comprises one or more sensors. In some embodiments, one of those sensor is an ECG sensor configured to (optionally continuously) monitor the heart rate. In some embodiments, as disclosed above, the data collected by the ECG sensor is optionally further processed, for example by a controller, and are optionally used as an input to decision making processes in device 100.

In some embodiments, as mentioned above, the device 100 comprises a memory that can be accessed it by the controller. In some embodiments, normal ECG morphology readings are stored in the memory and are used for the monitoring of the ECG morphology by comparing the current ECG morphology with the pre-stored normal ECG morphology.

In some embodiments, once a result of the comparison between the current ECG morphology and the pre-stored normal ECG morphology shows a discrepancy, the device 100 comprises instructions to activate one or more of the ICD and/or any other modality.

In some embodiments, ECG morphology evaluation is a process that might consume battery power, for example due to the high use of the evaluation process. In some embodiments, in order to save battery power, the controller comprises instructions to perform the ECG morphology evaluation according to one or more of the following parameters: a timing of the ECG morphology evaluation and a duration of the signal sampling window of the ECG morphology evaluation.

### Exemplary Timing of the ECG Morphology Evaluation

In some embodiments, the controller comprises instructions to perform the ECG morphology evaluation at determined time windows. A potential advantage of performing the ECG morphology evaluation at certain time windows is that it potentially reduces the battery power consumption of the device. In some embodiments, ECG morphology evaluation is performed at one or more of the following determined time windows:
1. During provision of stimulation periods, usually predetermined periods, and
2. When heart rate is within Ventricular Tachycardia periods (Ventricular Tachycardia is typically set when heart rate is above 100 beats per minute (BPM)), for example 120 BMP, 150 BMP, 175 BMP, 200 BPM, 300 BPM.
3. In some embodiments, optionally, when an atrial electrode is implanted and configured for sensing atrial contractions and therefore P-waves are monitored, morphology evaluation is performed when the Atrial-Ventricle (A-V) timing delay is above a set threshold. For example, a threshold is set as a fixed value, for example greater than 180 mS and/or, for example, a change of more than 10 mS from baseline measured A-V delay.
4. In some embodiments, optionally, when there is one electrode configured to sense R-wave in the right ventricle and another electrode configured to sense R-wave in the left ventricle, morphology evaluation is performed when the Right ventricle-Left ventricle (V-V) timing delay is above a set threshold. For example, threshold can be set as a fixed value greater than 10 mS or a change of more than ImS from baseline measured V-V timing delay.
5. In some embodiments, optionally, in relation to atrial tachycardia, the P-wave (atrial contraction) is monitored and in the case when a P-P delay is different from an R-R delay, then morphology evaluation may also be performed.

### Exemplary Duration of the Signal Sampling Window of the ECG Morphology Evaluation

In some embodiments, the controller comprises instructions to perform the ECG morphology evaluation at determined duration time windows. In some embodiments, the duration of the sampling time windows are decided according to one or more of the following parameters:
1. Short sampling time windows, for example up to 500 mS (for example 50 mS), during stimulation periods, optionally during predetermined stimulation periods.
2. Short sampling time windows, for example up to 500 mS morphology evaluation is performed (for example 50 mS), when a sensed A-V timing delay is above a set threshold. For example, a threshold is set as a fixed value, for example greater than 180 mS and/or, for example, a change of more than 10 mS from baseline measured A-V delay.
3. Short sampling time windows, for example up to 500 mS (For example 50 mS), when a sensed V-V timing delay is above a set threshold. For example, threshold can be set as a fixed value greater than 10 mS or a change of more than 1 mS from baseline measured V-V delay.
4. Long sampling time windows, for example longer than 200 mS (for example 2000 mS) when ventricular tachycardia is identified. In some cases the sampling is done during the whole period of ventricular tachycardia.

### Exemplary Stimulations

In some embodiments, the exemplary device provides one or more of the following stimulations ICD, Cardiac contractility modulation and CRT.

### Exemplary Methods

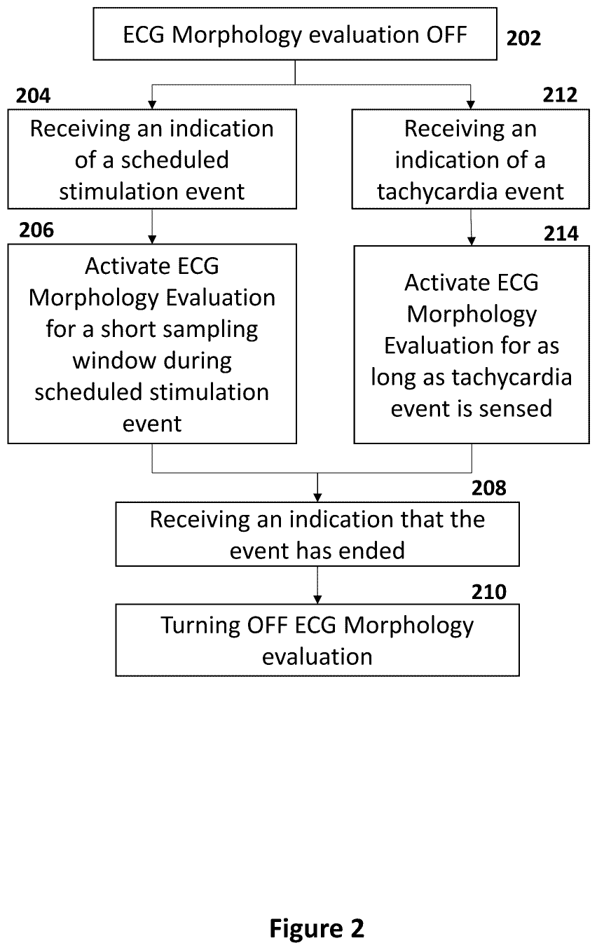

Referring now to FIG. 2, showing an exemplary method of performing morphology evaluation processes in an implantable device, according to some embodiments of the invention.

In some embodiments, the method begins with the ECG morphology evaluation OFF 202. In some embodiments, since the potential advantage of managing the ECG evaluation is to potentially reduce the battery power consumption, as a hypothetical beginning, the methods begin with the ECG morphology evaluation OFF. It should be understood that the method may begin at a point where the ECG morphology evaluation is ON.

In some embodiments, the controller receives an indication that a scheduled stimulation event is about to occur 204. In some embodiments, the controller comprises instructions to activate the ECG morphology evaluation for a short sampling window 206 (as explained above), for example for a sampling window of up to 50 mS.

In some embodiments, after activation of the ECG morphology evaluation, the system receives an indication that the event has ended 208, and then the system turns OFF the ECG morphology evaluation 210.

In some embodiments, the controller receives an indication that a tachycardia is occurring 212. In some embodiments, the controller comprises instructions to activate the ECG morphology evaluation for a long sampling window 214, for example, for as long as the tachycardia event is sensed, for example, until three or more consecutive readings are found to be in a normal predetermined range.

In some embodiments, the system then receives an indication that the tachycardia event has ended 208, and then the system turns OFF the ECG morphology evaluation 210.

In some embodiments, the system turns OFF the ECG morphology evaluation after a determined period of time, for example in the case of a stimulation event, the system will perform the ECG morphology evaluation during a predetermined period of time and will automatically turn OFF the ECG morphology evaluation without the need of receiving the indication that the event has ended.

As used herein with reference to quantity or value, the term “about” means “within ±20% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of performing morphology evaluation processes in an implantable device, the method comprising:
a. providing instructions to said implantable device for performing a morphology evaluation process at a determined timing according to predetermined parameters;
b. activating said morphology evaluation process for a predetermined period of time following sensing said predetermined parameters.

2. The method according to claim 1, wherein said predetermined parameters are one or more of a scheduled stimulation event and a tachycardia event.

3. The method according to claim 2, wherein said scheduled stimulation are one or more of Cardiac contractility modulation and CRT.

4. The method according to claim 2, wherein when said predetermined parameters is a scheduled stimulation event, then said predetermined period of time is at least one selected form the group consisting of:
a. up to 500 mS; and
b. up to 50 mS.

5. The method according to claim 2, wherein said tachycardia event is defined when a heart rate is above 100 beats per minute (BPM).

6. The method according to claim 2, wherein when said predetermined parameters is a tachycardia event, then said predetermined period of time is as long as said tachycardia event is sensed.

7. The method according to claim 2, wherein when said predetermined parameters is a tachycardia event, then said predetermined period of time is of equal or greater than 1000 mS.

8. The method according to claim 1, wherein said predetermined parameters are one or more of:
a. a detected atrial-ventricle (A-V) timing delay above a predetermined threshold;
b. a detected right ventricle-left ventricle (V-V) timing delay above a predetermined threshold; and
c. a detected P-P delay that is different from an R-R delay.

9. The method according to claim 8, wherein when said predetermined parameters is one or more of:
a. a detected atrial-ventricle (A-V) timing delay above a predetermined threshold;
b. a detected right ventricle-left ventricle (V-V) timing delay above a predetermined threshold; and
c. a detected P-P delay that is different from an R-R delay;
then said predetermined period of time is of up to 500 mS or up to 100 mS.

10. The method according to claim 8, wherein:
a. said predetermined threshold of said atrial-ventricle (A-V) timing delay is a sensed value above 180 mS;
b. said predetermined threshold of said atrial-ventricle (A-V) timing delay is a change of more than 10 mS from baseline measured A-V delay;
c. said predetermined threshold of said right ventricle-left ventricle (V-V) timing delay is a sensed value above 10 mS;
d. said predetermined threshold of said right ventricle-left ventricle (V-V) timing delay is a change of more than 1 mS from baseline measured V-V delay.

11. A system for performing morphology evaluation processes in an implantable device comprising an implantable controller, said controller configured to carry out the method of:
a. performing a morphology evaluation process at a determined timing according to predetermined parameters;
b. activating said morphology evaluation process for a predetermined period of time following sensing said predetermined parameters.

12. The system according to claim 11, wherein said predetermined parameters are one or more of a scheduled stimulation event and a tachycardia event.

13. The system according to claim 12, wherein said scheduled stimulation are one or more of Cardiac contractility modulation and CRT.

14. The system according to claim 12, wherein when said predetermined parameters is a scheduled stimulation event, then said predetermined period of time is at least one selected form the group consisting of:
a. up to 500 mS; and
b. up to 50 mS.

15. The system according to claim 12, wherein said tachycardia event is defined when a heart rate is above 100 beats per minute (BPM).

16. The system according to claim 12, wherein when said predetermined parameters is a tachycardia event, then said predetermined period of time is as long as said tachycardia event is sensed.

17. The system according to claim 12, wherein when said predetermined parameters is a tachycardia event, then said predetermined period of time is of equal or greater than 1000 mS.

18. The system according to claim 11, wherein said predetermined parameters are one or more of:
a. a detected atrial-ventricle (A-V) timing delay above a predetermined threshold;
b. a detected right ventricle-left ventricle (V-V) timing delay above a predetermined threshold; and
c. a detected P-P delay that is different from an R-R delay.

19. The system according to claim 18, wherein when said predetermined parameters is one or more of:
a. a detected atrial-ventricle (A-V) timing delay above a predetermined threshold;
b. a detected right ventricle-left ventricle (V-V) timing delay above a predetermined threshold; and
c. a detected P-P delay that is different from an R-R delay;
then said predetermined period of time is of up to 500 mS or up to 100 mS.

20. The system according to claim 18, wherein:
a. said predetermined threshold of said atrial-ventricle (A-V) timing delay is a sensed value above 180 mS;
b. said predetermined threshold of said atrial-ventricle (A-V) timing delay is a change of more than 10 mS from baseline measured A-V delay;
c. said predetermined threshold of said right ventricle-left ventricle (V-V) timing delay is a sensed value above 10 mS;
d. said predetermined threshold of said right ventricle-left ventricle (V-V) timing delay is a change of more than 1 mS from baseline measured V-V delay.
